(19) United States
(12) Patent Application Publication
(10) Pub. No.: US 2021/0161801 A1
(43) Pub. Date: Jun. 3, 2021

(54) SKIN CARE FILM BASED ON BIOFIBER

(71) Applicant: CHUANG SHENG MEDICINE EQUIPMENT CO. LTD., Taoyuan City (TW)

(72) Inventor: SUNG CHIH TING, Taoyuan City (TW)

(21) Appl. No.: 16/702,203

(22) Filed: Dec. 3, 2019

(51) Int. Cl. A61K 8/99; A61K 8/73; A61K 8/65; A61K 8/97; A61K 8/02

(52) U.S. Cl. CPC A61K 8/99; A61K 8/731; A61K 8/027; A61K 8/65; A61K 8/97; A61K 8/736

(57) ABSTRACT

A skin care film based on bioliber for promoting skin-repairing, skin-moisturizing and skin-soothing, including a bacterial-cellulose-type film body having a first surface, a second surface, which is opposite to the first surface, a functional particle storage layer, disposed on said first surface and containing a plurality of functional particles, a plurality of functional particle channels disposed throughout said bacterial-cellulose-type body film body and connect the said first surface with said second surface; and a plurality of functional particle inside-channel control-released layer provided on inner surface of said functional particle channel, said functional particles having a size smaller than the inner diameter of the functional particle channel and said functional particle inside-channel control-released layer is structured in such a way to assist controlled release of said functional particles that diffuse across said functional particle channels from said functional particle storage layer to said second surface.

### FIELD OF THE INVENTION

The present invention is based on a skin care film based on biofiber for promoting skin-repairing, skin-moisturizing and skin-soothing.

### BACKGROUND OF THE INVENTION

Traditionally, in addition to smear-like products, facial skin products have been gradually derived from the use of absorbable materials to impregnate functional ingredients to form mask products, while mask products can be generally classified into film-type mask, paste-type mask, gel-type mask, solid-type mask or emulsion-type mask. Recently, mask products are often expected to have various designs and functions with a basic effect of moisturizing, or whitening, or pull-up, or firming, or revitalizing, or deep cleaning, depending on clinical care needs.

The film-type mask has the advantages of being easy to carry and to use the mask product, and therefore the film-type mask is now often used as a portable and convenient way for daily facial beauty care or daily skin care. Conventional film-type masks generally comprise a film carrier and a composition that is adsorbed in the film carrier and used for skin care. The skin care composition is either a solution or gel state consisting of various functional compounds that have moisturizing, anti-oxidant, rejuvenating, lifting, firming and whitening effects for the face.

With the improvement of living standards, people's consumption demand and demand for cosmetics are also rising, skin care products have become an indispensable part of people's lives. A wide range of skin care products as a one-off beauty mask, set moisturizing, whitening, skin repairing in one multi-function products, due to the use of simple, easy to carry, consumer market is very broad. The conventional surface/eye/function pleural single substrate, normally carries only acting substance, rather than have an effect of improving the absorption rate of skin nutrients; and it is also inferior in transdermal skin surface, it tends to cause skin irritation, and may even lead to allergies, not suitable for long-term use.

Furthermore, it is widely used for post-operative maintenance, postoperative care, postoperative temperature adjustment, postoperative cooling, and other clinical demands. Furthermore, with the improvement of the level of people' life, the demand for beauty, the concept of skin care and maintenance, not only focus on the face, but also focus on other parts of the body such as breast and nipple, and believe that skin care in the other parts is important. However, the current market for the target is very little.

In view of the above reasons, products that are in good condition for daily maintenance and clinical care that meet the needs of different parts of the body are of major importance.

### SUMMARY OF THE INVENTION

The present invention is based on a skin care film based on biofiber for promoting skin-repairing, skin-moisturizing and skin-soothing, comprising a bacterial-cellulose-type film body having a first surface, a second surface, which is opposite to the first surface, a functional particle storage layer disposed on said first surface and containing a plurality of functional particles, a plurality of functional particle channels disposed throughout said bacterial-cellulose-type body film and connect the said first surface with said second surface; and a plurality of functional particle inside-channel control-released layer provided on inner surface of said functional particle channel, wherein said functional particles having a size smaller than the inner diameter of the functional particle channel and said functional particle inside-channel control-released layer is structured in such a way to assist controlled release of said functional particles that diffuse across said functional particle channels from said functional particle storage layer to said second surface.

Preferably, said functional particles comprise a mixture containing a plurality of centella asiatica extract type particles, a plurality of Irish moss extract type particles, and a plurality of aloe extract type particles.

Preferably, said functional particles have a good compatibility with said bacterial-cellulose-type film body.

Preferably, the bacterial-cellulose-type body has a thickness of 0.2 to 5 mm.

Preferably, the said functional particle storage layer has a thickness of 0.2 to 5 mm.

Preferably, the said functional particle has a particle diameter of 0.001 to 0.01 mm.

Preferably, the a bacterial-cellulose-type film body utilizes a material selected from the group consisting of non-woven fibers, wood pulp fibers, cotton fibers, silk fibers, collagen, chitin, polysaccharides, protein fiber, peptide fiber, bio-cellulose, acetic bio-cellulose, bacterial cellulose, sodium alginate, agar, natural fiber and its mixture.

Preferably, the said skin care film based on biofiber has a shape of a circle, a ring, a square, a ring with an inside hole of eye's profile, a ring with an inside hole of ear's profile, a ring with an inside hole of nose's profile, a ring with an inside hole of mouth's profile, a ring with an inside hole of a breast's profile, a ring with an inside hole of a nipper's profile, and a circle having a multiple of inside holes selected from a group of an eye's profile, an ear's profile, a nose's profile, a mouth's profile and their mixture.

### BRIEF DESCRIPTION OF THE DRAWING

The invention will be explained in conjunction with an illustrative embodiment shown in the accompanying drawing, in which:

FIG. 1 is a schematic illustration showing the skin care film based on biofiber for promoting skin-repairing, skin-moisturizing and skin-soothing.

FIG. 2 is a schematic shape for the clinical application of the skin care film based on biofiber for promoting skin-repairing, skin-moisturizing and skin-soothing: (A) skin care surrounding eyes, (B) skin care on a target microenvironment of body, (C) skin care surrounding month, and (D) skin care surrounding beast.

### DETAILED DESCRIPTION OF PREFERRED EMBODIMENTS

The present invention concerns a new skin care film based on biofiber for promoting skin-repairing, skin-moisturizing and skin-soothing, wherein the said skin care film based on biofiber comprising particularly suitable for women after medical skin care treatments. The skin care film based on biofiber of present invention comprises a mixture containing a plurality of centella asiatica extract type particles, a plurality of Irish moss extract type particles, and a plurality of aloe extract type particles; wherein said functional particles have a good compatibility with said bacterial-cellulose-type film body for tissue and skin repairing after medical treatments.

### Embodiment I: A New Skin Care Film Based on Biofiber for Promoting Skin-Repairing, Skin-Moisturizing and Skin-Soothing

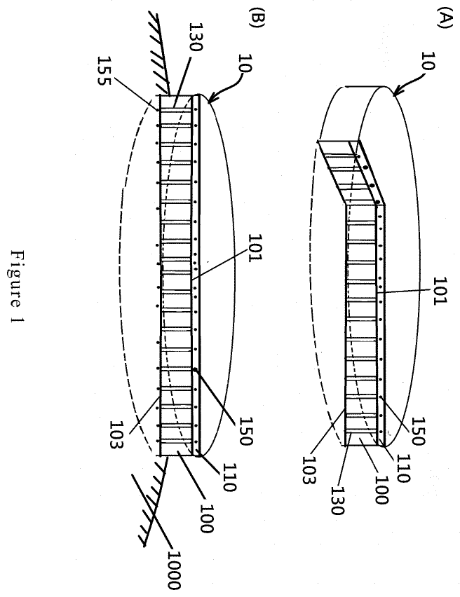

Reference is now made to FIGS. 1(A) and 1(B) showing a schematic illustration of a skin care film based on biofiber for promoting skin-repairing, skin-moisturizing and skin-soothing according to one embodiment I of the invention.

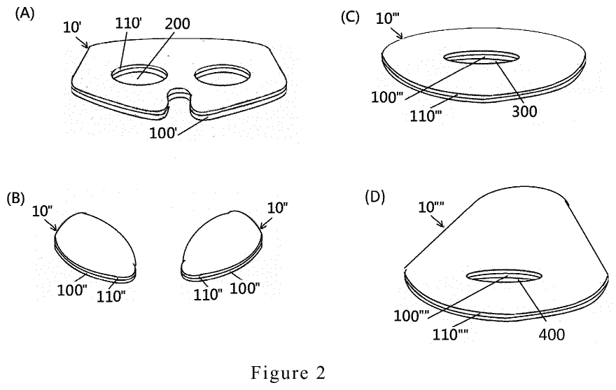

The skin care film based on biofiber 10 of said embodiment I, in accordance with the present disclosure comprises: a bacterial-cellulose-type film body 100 having a first surface 101, a second surface 103, which is opposite to the first surface 101, and a plurality of functional particle channels 130, which runs through the said bacterial-cellulose-type film body 100 and connect the said first surface 101 with said second surface 103; a plurality of functional particle inside-channel control-released layer 135, and a functional particle storage layer 110 disposed on said first surface 101 and containing said functional particles 150, wherein said functional particles 150 having a size smaller than the inner diameter of the functional particle channel 130 and comprise a mixture containing a plurality of centella asiatica extract type particles, a plurality of Irish moss extract type particles, and a plurality of aloe extract type particles; wherein said functional particles 150 have a good compatibility with said bacterial-cellulose-type film body 100. Said functional particle storage layer 110 was consisted of a selected group from a coated solution-type layer, a coated solid-type layer, a coated-type layer and its mixtures. The said functional particle 150 was consisted of a selected group from a micro-particle, a micro-capsule, a micro-gel, a micro-micelle, a micro-liposome, a micro-complex particle, and its mixtures. In general, said centella asiatica extract type particles, said Irish moss extract type particles, and said aloe extract type particles are buried inside said functional particle 150. In the clinical application, said centella asiatica extract type particles, said Irish moss extract type particles, and said aloe extract type particles would be released from said functional particle 150. Said centella asiatica extract type particles contains centella asiatica extracts, which were incorporated into the Taiwan and China pharmacopoeia, wherein in addition to wound healing, it was recommended for the treatment of various skin conditions such as leprosy, lupus, varicose ulcers, eczema, psoriasis, diarrholea, fever, amenorrhea, and diseases of the female genitourinary tract. Said Irish moss extract type particles contains Irish moss extracts, which provide a high fiber content in Irish Moss exerts a mild laxative effect whilst soothing inflamed tissues within the intestinal tract, potentially providing relief from a whole host of intestinal disorders. Its demulcent properties promote healthy digestion—soothing the films of the digestive tract. The gelatin like content helps to ease gastritis, dyspepsia, nausea, heartburn and indigestion. Said aloe extract type particles contains aloe extracts, which promote moisturizing and anti-aging effect. in the skin, aloe stimulates fibroblast which produces the collagen and elastin fibers making the skin more elastic and less wrinkled. Preferably, the bacterial-cellulose-type film body 100 has a thickness of 0.2 to 5 mm. Preferably, the said functional particle storage layer 110 has a thickness of 0.2 to 5 mm. Preferably, the said functional particle 150 has a particle diameter of 0.001 to 0.01 mm. Preferably, the a bacterial-cellulose-type film body 100 utilizes a material selected from the group consisting of non-woven fibers, wood pulp fibers, cotton fibers, silk fibers, collagen, chitin, polysaccharides, protein fiber, peptide fiber, bio-cellulose, acetic bio-cellulose, bacterial cellulose, sodium alginate, agar, natural fiber and its mixture. Preferably, the said skin care film based on biofiber 10 could be designed to various skin care films based on biofiber (10′, 10″, 10′″, and 10″″) (FIG. 2) with a clinical inside hole 105 (FIG. 1) depending on different clinic applications, which need a shape of a circle, a ring, a square, a ring with an inside hole of eye's profile, a ring with an inside hole of ear's profile, a ring with an inside hole of nose's profile, a ring with an inside hole of mouth's profile, a ring with an inside hole of a breast's profile, a ring with an inside hole of a nipper's profile, and a circle having a multiple of inside holes selected from a group of an eye's profile, an ear's profile, a nose's profile, a mouth's profile and their mixture.

### Embodiment II: A Skin Care Film Based on Biofiber with Various Shapes for Promoting Skin-Repairing, Skin-Moisturizing and Skin-Soothing

FIG. 2 is a schematic shape for the Embodiment II of the skin care film based on biofiber with various shapes for promoting skin-repairing, skin-moisturizing and skin-soothing: (A) skin care surrounding eyes, (B) skin care on a target microenvironment of body, (C) skin care surrounding month, and (D) skin care surrounding beast. The said skin care films based on biofiber are designed with various shapes (10′, 10″, 10′″, and 10″″) depending on different clinic applications, which need a shape of a circle, a ring, a square, a ring with an inside hole of eye's profile, a ring with an inside hole of ear's profile, a ring with an inside hole of nose's profile, a ring with an inside hole of mouth's profile, a ring with an inside hole of a breast's profile, a ring with an inside hole of a nipper's profile, and a circle having a multiple of inside holes selected from a group of an eye's profile, an ear's profile, a nose's profile, a mouth's profile and their mixture. Said functional particles 150 (structure referred to FIG. 1) in said functional particle storage layers 110, 110′, 110″, 110′″ could run through said functional particle channel 130 (structure referred to FIG. 1) to target skin 1000. Said centella asiatica extract type particles, said Irish moss extract type particles, and said aloe extract type particles would be released from said functional particle 150 (structure referred to FIG. 1).

While in the foregoing specification I have described some embodiments of my invention in considerable detail for the purpose of illustration, it is to be understood that many of the details herein given may be varied considerably by those skilled in the art without departing from the spirit and scope of my invention.

### REFERENCE NUMERALS OF FIGS.

10, 10′, 10″, 10′″, 10″″ a skin care film based on bio fiber
100, 100′, 100″, 100′″, 100″″ a bacterial-cellulose-type body film
101 a first surface
103 a second surface
105 a clinical inside hole
110, 110′, 110″, 110′″, 110″″ a functional particle storage layer
130 a functional particle channel
150 a functional particle
155 a released functional particle
200 an inside hole of eye's profile
300 an inside hole of ear's profile
400 an inside hole of nipper's profile
1000 a target skin

1. A skin care film based on biofiber for promoting skin-repairing, skin-moisturizing and skin-soothing, comprising:
a bacterial-cellulose-type film body having
a first surface,
a second surface, which is opposite to the first surface,
a functional particle storage layer disposed on said first surface and containing a plurality of functional particles,
a plurality of functional particle channels disposed throughout said bacterial-cellulose-type body film body and connect the said first surface with said second surface; and
a plurality of functional particle inside-channel control-released layer provided on inner surface of said functional particle channel,
wherein said functional particles having a size smaller than the inner diameter of the functional particle channel and said functional particle inside-channel control-released layer is structured in such a way to assist controlled release of said functional particles that diffuse across said functional particle channels from said functional particle storage layer to said second surface.

2. The skin care film based on biofiber according to claim 1, wherein said functional particles comprise a mixture containing a plurality of centella asiatica extract type particles, a plurality of Irish moss extract type particles, and a plurality of aloe extract type particles.

3. The skin care film based on biofiber according to claim 1, wherein said functional particles have a good compatibility with said bacterial-cellulose-type film body.

4. The skin care film based on biofiber according to claim 1, wherein the bacterial-cellulose-type body has a thickness of 0.2 to 5 mm.

5. The skin care film based on biofiber according to claim 1, wherein the said functional particle storage layer has a thickness of 0.2 to 5 mm.

6. The skin care film based on biofiber according to claim 1, wherein the said functional particle has a particle diameter of 0.001 to 0.01 mm.

7. The skin care film based on biofiber of claim 1, wherein the a bacterial-cellulose-type film body utilizes a material selected from the group consisting of non-woven fibers, wood pulp fibers, cotton fibers, silk fibers, collagen, chitin, polysaccharides, protein fiber, peptide fiber, bio-cellulose, acetic bio-cellulose, bacterial cellulose, sodium alginate, agar, natural fiber and its mixture.

8. The skin care film based on biofiber of claim 1, wherein the said skin care film based on biofiber has a shape of a circle, a ring, a square, a ring with an inside hole of eye's profile, a ring with an inside hole of ear's profile, a ring with an inside hole of nose's profile, a ring with an inside hole of mouth's profile, a ring with an inside hole of a breast's profile, a ring with an inside hole of a nipper's profile, and a circle having a multiple of inside holes selected from a group of an eye's profile, an ear's profile, a nose's profile, a mouth's profile and their mixture.
